(19) United States
(12) Patent Application Publication
(10) Pub. No.: US 2009/0204175 A1
(43) Pub. Date: Aug. 13, 2009

(54) ELECTROSTIMULATING APPARATUS AND METHOD

(75) Inventors: Andrea Zanella, Mirandola (IT); Guido Comai, Budrio (IT); Alessandro Zanna, Bologna (IT); Massimo Barrella, Roma (IT); Rosanna Toscano, Parabiago (IT)

Correspondence Address: NIXON & VANDERHYE, PC, 901 NORTH GLEBE ROAD, 11TH FLOOR, ARLINGTON, VA, 22203, US

(73) Assignee: LORENZ BIOTECH S.P.A., Medolla (IT)

(21) Appl. No.: 12/293,236

(22) PCT Filed: Mar. 15, 2007
(86) PCT No.: PCT/IB2007/000637

§ 371 Date: Nov. 18, 2008

(30) Foreign Application Priority Data
Mar. 17, 2006 (IT) MO2006A000087

(51) Int. Cl. A61N 1/00

(52) U.S. Cl. 607/48; 607/72

(57) ABSTRACT

An electrostimulating apparatus comprises a generating arrangement for generating electric pulses organised in sequences having preset values of typical parameters, the typical parameters comprising amplitude, width and frequency of the pulses, a plurality of stimulation channels such as to dispense the sequences to body zones of an organism in an independent manner, a varying arrangement suitable for varying at least one of the typical parameters in such a way as to substantially prevent the organism from habituating to the electric pulses; a method for electrostimulating an organism comprises: producing a sequence of electric pulses having a relaxing effect and a further sequence of electric pulses having a vasoactive effect; dispensing the sequence to body zones of the organism, and further dispensing the further sequence to further body zones of the organism, the body zones and the further body zones comprising agonist muscles and antagonist muscles of a neuromuscular compartment comprised in the organism.

### CROSS-REFERENCE TO RELATED APPLICATIONS

This application is the U.S. national phase of International Application No. PCT/IB2007/000637, filed 15 Mar. 2007, which designated the U.S and claims priority to Italy Application Nos. MO2006A000087, filed 17 Mar. 2006, the entire contents of each application is hereby incorporated by reference.

### DESCRIPTION

The invention relates to an electrostimulating apparatus and method.

In neurophysiology, the H reflex, or Hoffman reflex is known, which, although it is a reflex that is very similar to the monosynaptic reflex following a mechanical stretching of a muscle, may also be evoked through an electric stimulation conducted at the level of an afferent innervation. In the recent past, the H reflex in humans has been studied widely, as the features of the latter enable useful information to be obtained for defining the spinal excitability in humans both in physiological and pathological conditions. In particular, the modulation of the H reflex has been studied following serious clinical manifestations of a heterogeneous group of pathologies, comprising spasticity, dystonia and fibromyalgia. In these pathologies, an increase in spinal excitation at the level of a single metamer or of several metamers is recognised as a physiopathological common denominator that is activated by various central and peripheral influences, and the spinal excitation can be studied in vivo in humans by evaluating carefully the H reflex both in terms of latency and in terms of the amplitude of the reflex with respect to the dispensed stimulation. The H reflex is definable as the simplest of the spinal reflexes and can be evoked by electrically stimulating type Ia afferent fibres comprised in the muscle spindle endings. This stimulation is followed by a transmission of the evoked discharge afferent to the spinal cord, a production of a synchronised postsynaptic excitatory potential that is sufficient to discharge the motor neurons of a relevant pool with a transmission of the reflex discharge along the axons of the alpha-type motor neurons to the muscle. The excitability of the spinal motor neuron depends directly on the descending central path under the systemic influence, which is typically at the endocrine level and is mediated by circulating neurotransmitters, of projection of the peripheral reflex arch. The measurement of the minimum latency of the H wave, combined with the amplitude, width and threshold values of the latter, provides information on the conduction level of the reflex arch. The amplitude of the H reflex on the other hand enables to measure indirectly the quantity of alpha motor neurons that have been activated synchronously, modulated by various afferences. A weak voluntary contraction strengthens the H reflex, increasing the discharge of the motor neuron pool, but alters the latency of the reflex. In non-pathological circumstances, the H reflex can be recorded from the soleus muscle by stimulating the tibial nerve and from the flexor carpi radialis muscle by stimulating the median nerve through a low-frequency stimulus.

If it is not possible to reproduce a reflected response this can be ascribed to an afferent disturbance or to a low central excitability. The low central excitability does not necessarily indicate a specific pathology, as the test during a weak muscular contraction may reveal an intact reflex path with a normal latency. In the literature, there are reported various attempts to reduce the hyperexcitability of the motor neuron through Transcutaneous Electric Stimulation (TENS), although there is no univocal consensus on the effect that the latter could have on the Hoffman reflex. The spinal excitability is regulated by many influences that can be concisely classified as above the spinal cord, systemic (due to hormones and circulating neurotransmitters), propriospinal (intra-spinal connections) or reflected peripheral influences.

The reflected peripheral influences in turn comprise a combination of reflex arches, which are both monosynaptic and oligo- or multisynaptic and are integrated at a distinct spinal innervation level (metamer). The peripheral afferences come from the central branch of the cells of the spinal ganglia. The peripheral branch is connected to different types of receptor: the muscle spindles, the tendon receptors, the joint receptors and various types of cutaneous receptors. In particular, the afferences of the muscle spindles (fibres Ia) are the afferences that determine the most direct relations with the pool of the alpha motor neurons interacting in the so-called “Sherrington monosynaptic reflex”. Although the Sherrington reflex model is still an object of discussion, it can be stated that when a muscle is stretched the primary sensory fibres, i.e. the afferent neurons of the group Ia of the muscle spindles, respond both to the speed and degree of extension, sending the information at the spinal level. On the other hand, the secondary sensory fibres, i.e. the afferent neurons of the group 11, detect and send to the central nervous system (CNS) only the information relating to the degree of stretching. This information is transmitted monosynaptically to the alpha motor neuron that activates the extrafusal fibres in order to reduce the stretching and is transmitted polysynaptically, by means of an interneuron, to another alpha motor neuron that inhibits the contraction in the antagonist muscle. Further, at the same time, through two types of gamma motor neurons, known as static and dynamic motor neurons, the CNS is able to influence the afferences of the muscle spindles during movement. The muscle spindle is thus definable as the most important proprioceptor, having a fundamental role in the movement and the control of the reflex activity. The combined signal coming from a plurality of muscle spindles of each muscle provides the CNS with information, generating a fine adjustment of the muscular activation and thus acting as a sort of servo control. At the same time, the muscle spindles are controlled in a continuous manner by the gamma neurons that the CNS controls separately from the alpha motor neurons by controlling all muscle functions. The intrafusal fibres are typically excited by the stimulation below the extrafusal motor threshold: as soon as the motor threshold has been exceeded, the muscle contraction activates the tendon receptors, which provoke the effect of the muscle spindles.

WO 02/09809 discloses an apparatus for treating muscular, tendon and vascular pathologies by means of which a stimulation is applied to a patient, which stimulation comprises a series of electric pulses having a width comprised between 10 and 40 microseconds and an intensity that is variable in function of the impedance and conductance of the tissue subjected to stimulation, and comprised between 100 and 170 microamperes.

WO 2004/084988 discloses an electrostimulating apparatus owing to which it is possible, in function of the type of electric stimulation produced and of the configuration parameters adopted, to generate an induced bioactive neuromodulation, which is suitable for producing vasoactive phenomena on the microcircle and on the macrocircle. These phenomena are in turn mediated by phenomena connected to the direct stimulation of the smooth muscle and by essentially catecolaminergic phenomena, by means of stimulation of the postsynaptic receptors. The aforesaid apparatus is able to produce specific stimulation sequences that induce reproducible and constant neurophysiological responses. In particular, WO 2004/084988 discloses an activating sequence for activating the microcircle (ATMC) and a relaxing sequence for relaxing the muscle fibre (DCTR), which are able to stimulate various functional contingents, including the striated muscle, the smooth muscle and the peripheral mixed nerve. The aforesaid stimulation sequences are assembled on three basic parameters: the width of the stimulation, the frequency of the stimulation and the intervals of time during which different width/frequency combinations follow. The general operating model of the stimulation sequences reflects the digital-analogue transduction that occurs in the transmission of a nerve pulse.

The neuronal electric stimulation by modulation of frequency and amplitude, or FREMS™ (Frequency Rhythmic Electric Modulation System™), disclosed in the aforesaid WO 2004/084988 and in WO 2004/067087 (incorporated herein for reference), is characterised by the use of transcutaneous electric currents, which are produced by means of sequential electric pulses having variable frequency and width. The frequency may vary between 0.1 to 999 Hz, the width of the stimulation is comprised between 0.1 and 40 μs and the voltage, which is kept constantly above the perception threshold, is comprised between 0.1 and 300 V (preferably 150 V). By suitably combining the aforesaid frequency and width variations a specific sequence defined as DCTR is obtained, having a relaxing effect and comprising a series of subphases, called A, B and C. Frequency and width are constant in the subphase A, the frequency is constant and the width is variable in the subphase B, the frequency is variable and the width is constant in the subphase C.

Experimental studies have enabled the effects of FREMS to be evaluated and the capacity of the latter to evoke compound muscle action potentials (cMAP) to be evaluated, which are obtainable in the adductor hallucis muscle by stimulating the posterior tibial nerve, as well as the variation in amplitude of the aforesaid H reflex by using the latter as a conditioning stimulus. As disclosed in WO 2004/084988, the aforesaid experimental studies have also shown that the greatest amplitude of the cMAPs that is obtainable (0.60±0.02 mV) is approximately 15 times less than that of the cMAPs obtained with the known devices that dispense TENS current, i.e. amplitudes of the order of 9±0.6 mV with stimuli having a width typically comprised in a range of 200-1000 μs. It has been further observed that the maximum amplitude value of the cMAPs is obtained in the presence of a width/frequency ratio of 0.13 (40 μs/29 Hz).

A further type of sequence, called ATCM and suitably designed for obtaining a vasoactive effect, has a prevailing action on the motility of the microcircle, i.e. of the smooth sphincters of the arterioles and venules of the subcutaneous tissue. The ATCM sequence is divisible into three subsequences, called S1, S2, S3. The subsequences S1 and S3 are both distinguished by a frequency increase phase, with distinct time modes. The subsequence S2 is mainly constituted for producing variability in the width of the individual stimuli, in a gradually increasing frequency range, in such a way as to reduce the bioreaction, until the latter is stabilised. More in detail, the subsequence S1, having a relaxing effect and therefore having an effect that is very similar to the aforesaid DCTR sequence, comprises phases in which, after a first adaptation phase conducted at 1 Hz frequency, the frequency is gradually increased at a constant amplitude, thus decreasing the bioreaction in a gradual manner. Subsequently, the frequency is increased in a much more rapid manner until it reaches a target of 19 Hz. The subsequence S2 is then run, which is in turn divisible into four phases, called S2-A, S2-B, S2-C and S2-D. In the subsequence S2, after a phase (S2-A) conducted at a constant frequency in which the amplitude is rapidly increased until the instant 1, the frequency is gradually increased and consequently the bioreaction rapidly falls until the instant 2 (S2-B). At this point the amplitude is reset that again rises at a constant frequency until the instant 3 (S2-C). Subsequently, the frequency again increases gradually whilst the amplitude is kept constant and, consequently, the bioreaction gradually decreases until the instant 3 (S2-D). In this way the bioreaction is varied in a discontinuous manner, producing points of sudden slope variation, i.e. the points 1, 2 and 3. In practice, as disclosed in WO 2004/084988, a system is obtained producing a sequence of vasodilations and vasocontractions with sequential increases and decreases of haematic flow of the microcircle surrounding the stimulation zone. These vasodilations and vasocontractions produce a “pump” effect that is clearly produced by neuromodulation of the sympathetic neurovegetative system, which influences the vasoaction through the smooth muscle of the capillary vessels and the arterioles. In this way it can be shown that this subsequence, which is distinguished by alternating variations of the rheobase, therefore produces a vasoactive effect consisting of sequential vasodilation phases and vasoconstriction phases. This definitely produces a draining effect and, above all, makes the microcircle elastic and modulates the latter around a main carrying event that determines the average variation thereof.

An object of the invention is to improve known electrostimulating apparatuses.

Another object is to provide an electrostimulating apparatus that enables muscular hyperexcitability of spinal and/or cerebral origin in a patient to be treated.

A further object is to provide an electrostimulating apparatus and method that enables muscular hyperexcitability of spinal and/or cerebral origin in a patient to be treated.

In a first aspect of the invention, there is provided an electrostimulating apparatus, comprising a generating arrangement for generating electric pulses organised in sequences having preset values of typical parameters, said typical parameters comprising amplitude, width and frequency of said pulses, a plurality of stimulation channels such as to dispense said sequences to body zones of an organism in an independent manner, a varying arrangement suitable for varying at least one of said typical parameters so as to substantially prevent said organism from habituating to said electric pulses.

In a second aspect of the invention, there is provided a method for electrostimulating an organism, comprising:
producing a sequence of electric pulses having a relaxing effect and a further sequence of electric pulses having a vasoactive effect;
dispensing said sequence to body zones of said organism, and further dispensing said further sequence to further body zones of said organism, said body zones and said further body zones comprising an agonist muscle and an antagonist muscle of a neuromuscular compartment comprised in said organism.

These aspects of the invention are based on a new neurophysiological effect that was found during recent experimental studies conducted on the aforesaid FREMS. These studies have in fact shown that the amplitude of the H reflex sampled from the ipsilateral soleus muscle with or without conditioning of the FREMS applied to the short adductor hallucis muscle, is substantially decreased (by a value equal to 50%) during FREMS stimulation. The amplitude variation of the H reflex is significantly influenced by the variations of the width pulse/stimulation frequency ratio (w/f), in particular during the subphase C (r²=0.43; p<0.001). This result suggested that the FREMS is actually capable of modulating the amplitude of the H reflex, very probably through active recruitment of the muscle spindles.

Owing to these results it has been possible to make a new electrostimulating apparatus, by means of which a new electrostimulating method can be carried out for treating the spinal hyperexcitability that is secondary to cerebral or spinal damage and is the cause of spasticity in a patient. The new electrostimulating apparatus enables the aforesaid FREMS to be applied, with different sequences and simultaneously, in two antagonist neuromuscular districts of a motor limb that are connected to the same metamer and mutually connected through an afferent neuron/interneuron/alpha motor neuron loop (circuit). In this way, a synergic effect can be produced that inhibits the hypertonic contraction, which contraction is typically caused by the dysfunctions of the upper motor neuron and is therefore typical of the spastic phenomena that are secondary to cerebral or spinal damage of the central nervous system.

The invention can be better understood and implemented with reference to the attached Figures, which illustrate an exemplifying but non-limiting embodiment thereof, in which:

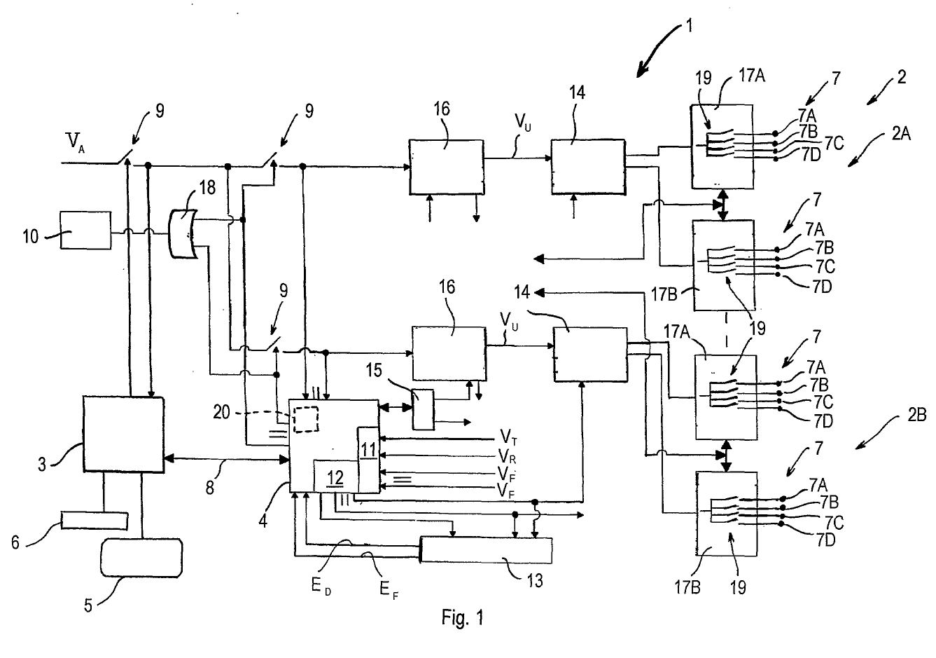

FIG. 1 is a block diagram illustrating an electrostimulating apparatus comprising a plurality of independent stimulation channels;

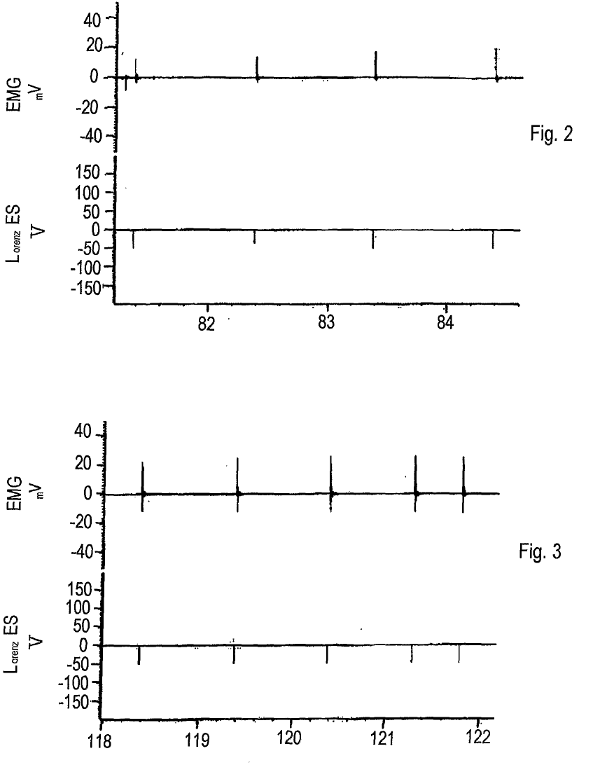

FIGS. 2 to 4 show electromyograms illustrating the production of cMAP in the abductor hallucis muscle obtained by stimulating the posterior tibial nerve with DCTR sequences;

FIG. 5 shows a potential difference/time Cartesian graph, illustrating the variation in the cMAP value during the subphases A, B and C of a DCTR sequence;

FIG. 6 shows a potential difference/ratio between pulse width and pulse frequency Cartesian graph, illustrating the variation in the cMAP value during the application of a DCTR sequence;

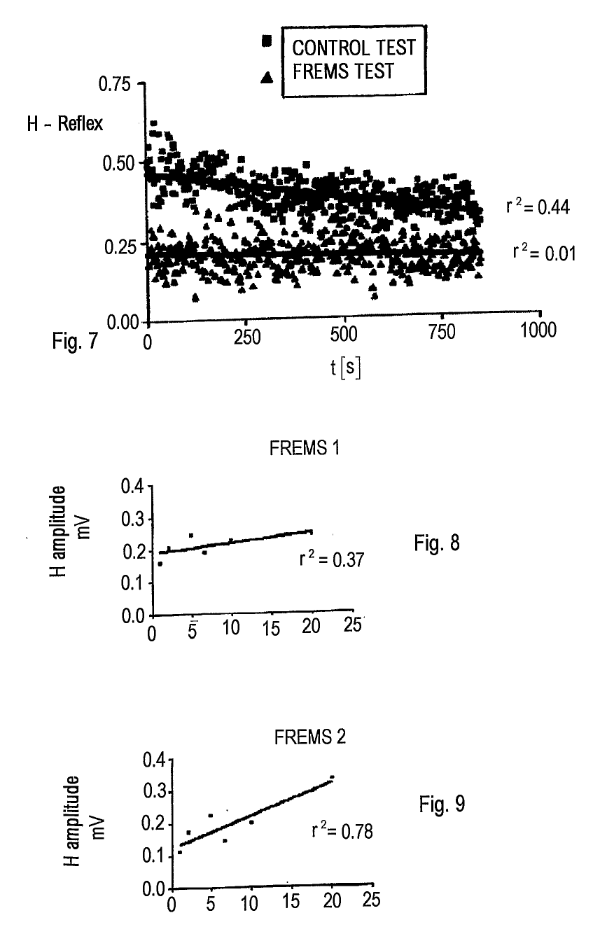

FIG. 7 is a graph illustrating the amplitude of the H reflex in the presence or absence of FREMS stimulation;

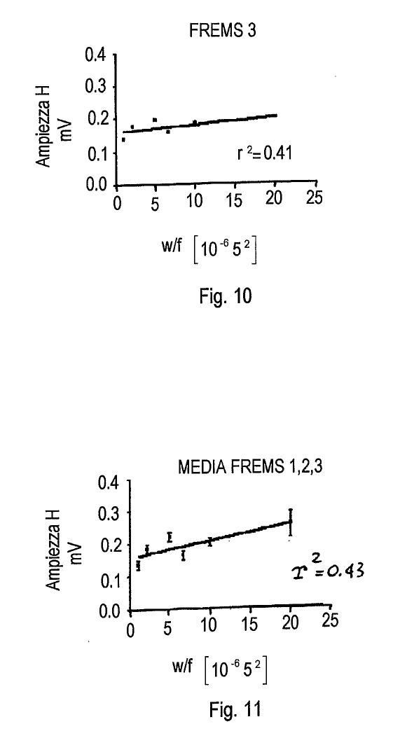

FIGS. 8 to 10 show Cartesian graphs illustrating the amplitude variation of the H reflex in function of the variation in the ratio between pulse width and pulse frequency, during three FREMS stimulation sessions;

FIG. 11 shows a Cartesian graph illustrating the average amplitude variations of the H reflex in function of the variations in the ratio between pulse width and pulse frequency, as measured during the three FREMS stimulations of FIGS. 8-10.

FIG. 1 shows schematically the assembly of the circuits comprised in an electrostimulating apparatus 1 that is able to produce and dispense the aforesaid DCTR (relaxing) sequences and ATMC (vasoactive) sequences comprised in FREMS stimulation through a plurality of independent stimulation channels 2, each of which is formed by a plurality of pairs of transcutaneous electrodes 7.

In the embodiment of the apparatus 1 shown in FIG. 1 there are provided four stimulation channels 2, of which only two are shown (for reasons of clarity) and are indicated by 2A, 2B.

In an embodiment that is not shown, there is provided an apparatus 1 comprising a number of stimulation channels 2 that is greater than four.

In another embodiment that is not shown, there is provided an apparatus 1 comprising a number of stimulation channels 2 that is less than four.

The apparatus 1 comprises a first control unit 3 and a second control unit 4, which interact with one another and are made of microprocessors of known type. The first control unit 3 controls a displaying device, for example a liquid crystal display 5, and an alphanumeric keyboard 6. By keying in on the latter a user of the apparatus 1 can direct the operation of the latter and set the parameters, which are displayable on the display 5, of the electric stimulations to be administered to a patient.

In an embodiment that is not shown, there is provided a remote-control device by means of which a patient connected to the apparatus 1 can control the operation of the latter without interacting with the keyboard 6. This embodiment is particularly useful inasmuch as it enables the patient to control the apparatus 1 by acting as a sensory feedback element relating to one or more operating parameters of the apparatus 1. The first control unit 3 controls a safety switch 9, which in turn controls an input supply voltage V_{A}. In normal operating conditions, the switch 9 is closed and a voltage adjuster 16 (the function of which will be disclosed below) that is comprised in each stimulation channel 2 is thus supplied. In emergency conditions, for example in the event of apparatus faults, the first control unit 1 opens the switch 9 and thus interrupts the supply to the voltage adjuster 16. To the second control unit 4 a luminous device, for example a LED 10 of known type, is further connected. When a patient is connected to the apparatus 1 by means of the electrodes 7 and the apparatus 1, supplied by the voltage V_{A}, administers an electric stimulation to the patient, the LED 10 lights up, thus indicating that the patient is subjected to the action of an electric current.

Through a serial communication interface 8, of known type, the first control unit 3 is connected to the second control unit 4, which controls the production of the electric pulses by adjusting the basic parameters thereof, i.e. amplitude, width and frequency, and comprises an analogue-digital converter (ADC) 11 and an integrated timing unit (ITU) 12. In the second control unit 4 there can be housed a support 20 (that is shown by means of a dotted line) on which the data are recorded that are necessary for the operation of the apparatus 1, such as, for example, the data relating to the stimulation sequences that are producible by the apparatus 1. The support 20 is readable through a data processing device (which is not shown), of known type, comprised in the apparatus 1 or arranged outside the apparatus 1 and interfaced with the latter. The data processing device, if it is comprised in the apparatus 1, may, for example, be positioned in the second control unit 4.

In an embodiment that is not shown, the support 20 is housed in the first control unit 3.

The analogue-digital converter 11 receives a feedback signal (in the form of voltage) relating to the pulse amplitude, and intervenes by producing an adjustment and/or an alarm signal if the pulse amplitude produced by the apparatus 1 is different from that set by the user. In particular, the analogue-digital converter 11 receives a reference voltage V_{T} regulating the operation of the analogue-digital converter 11, a further reference voltage V_{R}, which enables the correct operation of the analogue-digital converter 11 to be checked, and, from each of the stimulation channels 2, a feedback voltage V_{F}.

The integrated timing unit 12 defines the width and frequency of the pulse by interacting with a timing control device 13. The latter controls the width and frequency of the produced pulse and, if one or the other of these parameters is not correct, produces and sends a width error signal E_{D} and/or a frequency error signal E_{F}, which are able to arrest the second control unit 4.

Similarly to what has been disclosed in relation to the first control unit 3, also the second control unit 4 controls safety switches 9, which are provided in a number equal to the number of stimulation channels 2 comprised in the apparatus 1. The safety switches 9 controlled by the first control unit 3 and by the second control unit 4 interact with one another and with the LED 10 through an “OR”-type logic port 18.

The electric signals defining the frequency and width of the pulse are produced by the integrated timing unit 12 and are sent directly to an outlet pulses generator 14. In the apparatus 1 the outlet pulses generators 14 and the stimulation channels 2 are provided in equal numbers. Pulse width is defined and adjusted by a digital-analogue converter (DAC) 15 interacting with the second control unit 4. The digital-analogue converter 15 produces a plurality of electric signals defining the pulse amplitude for each single channel 2, and each signal is sent to a voltage adjuster 16. The apparatus 1 comprises a number of voltage adjusters 16 that is equal to the number of stimulation channels 2. An outlet voltage V_{U}, the value of which is comprised between 0 and 300 Volts, is produced by each voltage adjuster 16 and is sent to a corresponding outlet pulses generator 14. Each outlet pulses generator 14 produces a pulse having a preset, frequency and width and sends this pulse to a pair of outlet selectors 17A, 17B to which the electrodes 7 are connected. The pairs of outlet selectors 17A, 17B are provided in a number equal to the number of outlet pulses generators 14 comprised in the apparatus 1. Each outlet selector 17A, 17B comprises a plurality of switches 19, which are provided in a number equal to the number of electrodes 7 connected to the selector, by means of which switches the produced pulse can be alternatively transmitted to the corresponding electrode 7, or stopped. In each pair of outlet selectors 17A, 17B the electrodes 7 are associated functionally so as to form four pairs, the electrodes of each pair being indicated respectively as 7A, 7B, 7C and 7D. The electrodes 7 of each pair are connected to the corresponding outlet selector 17A or 17B.

In an embodiment that is not shown, outlet selectors 17A, 17B are provided comprising a number of pairs of electrodes 7 greater than four.

In another embodiment that is not shown, there are provided outlet selectors 17A, 17B comprising a number of pairs of electrodes 7 that are less than four.

When the apparatus 1 is in use, by acting on the switches 19, it is possible to select the electrodes 7 to which to send the pulse produced by the outlet pulses generators 14. It is thus possible to use independently both the pairs of electrodes 7A-7D comprised in two or more stimulation channels 2 and the pairs of electrodes 7A-7D comprised in a single stimulation channel 2.

As the second control unit 4, by means of the digital-analogue converter 15 and the integrated timing unit 12, is able to adjust the amplitude, width and frequency of the pulses produced in the stimulation channels 2 in an independent manner for each channel 2, the apparatus 1 is such as to be able to multiply the outlet pulses and space the latter in a preset manner.

Further, the integrated timing unit 12 enables the width of the outlet pulse to be increased in a preset manner. In particular, it is possible to obtain a percentage increase of the width of an electric stimulation pulse that is conducted in a plurality of phases, after the completion of which phases the width of the pulse remains constant. The percentage increase of the width of the pulse, the width of the pulse and the number of the phases are mutually correlated by the following formula:

Tᵢ(Nf)=T₀×(1+I%)^{Nf}

where:
Nf=Number of phase;
Tᵢ(Nf)=Width of stimulation pulse in function of the number of phase;
T₀=Width of initial stimulation pulse;
I %=Percentage increase of pulse width.

In the embodiment of the apparatus 1 illustrated in FIG. 1, the obtainable percentage increase I % is equal to 20%, 25%, 33%, 50%, and the values expressing Nf (i.e., the number of phases) is comprised between 0 and 9.

The integrated timing unit 12 further enables to vary in a pseudorandom manner the length of the period of time that elapses between two subsequent phases. In this way, it is possible to produce stimulation sequences in which the width of the pulses varies proportionately to the percentage increase in a random manner. This enables phenomena of biological accommodation to be prevented, i.e. the stimulated tissues in a patient are prevented from habituating to the pulses and thus becoming less sensitive to the latter.

In the embodiment of the apparatus 1 illustrated in FIG. 1, there are provided at least four periods of time that can be generated by random numbers.

In order to prevent the aforesaid phenomena of biological accommodation, the apparatus 1 can also act by varying the frequency and the amplitude of the pulses. The frequency, as previously disclosed, is adjusted by the integrated timing unit 12, whilst the amplitude is adjusted by the digital-analogue converter 15.

As previously disclosed, there is provided an embodiment of the apparatus 1 equipped with a remote control, by using which the patient may act as a sensory feedback element with respect to operation of the apparatus 1. In fact, the patient can be suitably instructed to vary the amplitude during the electrostimulating treatment by acting on the digital-analogue converter 15 through the remote control so as to prevent the aforesaid phenomena of biological accommodation. For example, the patient can be instructed to vary the pulse amplitude when the pulse reaches a maximum (subjective) level of tolerability. Alternatively, the patient can be instructed to vary the pulse amplitude when the pulse reaches the sensitivity threshold.

In use, the apparatus 1 is connected to a patient affected by spastic phenomena and at least two distinct stimulation channels 2 are used, for example the aforesaid channels 2A and 2B, the electrodes 7 of which are applied respectively to a body region near the specific efferent nerve of a hypertonic muscle (agonist muscle) and at a further body region comprising the corresponding antagonist muscle. The hypertonic muscle is then stimulated through the DCTR relaxing sequence whilst, simultaneously, the antagonist muscle is stimulated through the ATMC vasoactive sequence. The latter enables a direct muscular stimulation as well as an interaction with the sympathetic afferents and the afferents of the neurovegetative system, such as to close the circuit comprising motor neuron, interneuron and afferent neuron. The aforesaid double, simultaneous and differentiated stimulation inhibits the contraction of the hypertonic agonist muscle and rhythmically excites the motor neuron that is in synergy with the antagonist hypotonic muscle, creating mutual inhibition through the channel of the interneuron. The aforesaid effect of inhibition of the contraction of the hypertonic muscle is obtained by stimulating the latter with a sequence that is suitable for producing a phase depression of the H reflex.

When necessary, by using a suitable number of stimulation channels 2, and therefore a suitable number of pairs of electrodes 7, it is possible to stimulate simultaneously more than two body zones of the patient, in particular 4, 8 or 16 body zones. The pulses dispensed to the various body zones may or may not have the same frequency, and may be dispensed in a simultaneous manner or in a spaced over time, i.e. sequential, manner.

When the apparatus 1 is used to stimulate electrically a plurality of body zones of the patient, it is possible, during treatment, to select a certain number of body zones and limit the stimulation to the latter. This is obtained by acting on the second control unit 4 so as to exclude, for a preset period of time, all the stimulation channels 2 except for those relating to the body zones that it is desired to stimulate.

All the parameters relating to the operating modes of the apparatus 1, including the aforesaid “preferential zones” stimulation mode, can be recorded on the aforesaid support 20, which thus enables operation of the apparatus 1 to be programmed.

The experimental results are set out below that have led to the creation of the electrostimulating apparatus 1 disclosed above and the subsequent confirmations provided by the clinical experimentation.

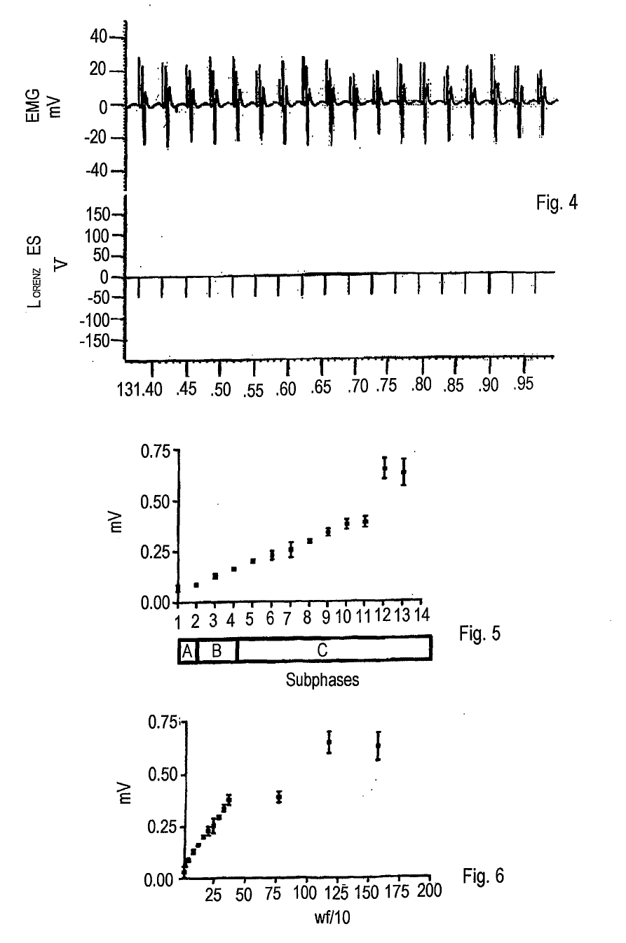

In order to verify the possibility of using the FREMS stimulation in the treatment of the muscular hyperexcitability of spinal and/or cerebral origin, sequences of electric pulses of the aforesaid DCTR-type were used that were produced by a Lorenz™ electrostimulating apparatus. In these DCTR sequences, the successive width variations (between 10 and 40 μs) and frequency variations (between 1 and 39 Hz) can induce compound action potentials (cMAP) if applied along the motor nerve of the muscle, in a similar way to what occurs with voluntary muscle recruitment by means of the temporal summation. In particular, it was wished to evaluate the possibility of influencing the motor spinal activity through a different regulation of the activation of the various types of muscle spindle. For this purpose, the variation of the amplitude of the H reflex was evaluated, which was obtained by evoking the latter between the soleus muscle and the abductor hallucis muscle, both partially innerved at the level of the first sacral vertebra (S1). As shown in FIGS. 4 to 6, it was possible to obtain the cMAPs in the abductor hallucis muscle by stimulating the posterior tibial nerve with DCTR sequences. The highest cMAP value, measured in terms of entire amplitude of the signal or RMS (0.60 mV±0.02), was about 15 times less than the amplitude of the cMAP obtainable with the electric stimulators TENS of known type, which use stimuli having a width of 200-1000 μs and produce cMAP the value of which is equal to approximately 9-10 mV. The maximum value of RMS amplitude of the cMAP is detectable in the presence of a w/f ratio equal to 0.13, a value that corresponds to a pulse frequency of 29 Hz and to a stimulus width equal to 40 μs. By increasing further the stimulation frequency up to 39 Hz, the w/f ratio falls to 0.10 and the value of RMS amplitude of the cMAP decreases slightly. As no correlation between the absolute value of the w/f ratio and the RMS amplitude of the cMAP can be shown, it can be assumed that the increase of the cMAP is connected to the progression of the DCTR sequence and not directly to the absolute value of the w/f ratio.

FIG. 7 shows the amplitude of the H reflex with or without FREMS stimulation. In absence of the latter, the H reflex decreases progressively with a significant linear correlation (r²=0.44). In the presence of FREMS stimulation, the amplitude of the H reflex decreases immediately and remains at low levels, but without showing any correlation (r²=0.01). This demonstrates the possibility of obtaining the modulation of the H reflex at the pulse frequency (f) and pulse width (w) variations, expressed as the w/f ratio. The results show that this pattern of stimulation induces a direct and reproducible modulation of the excitability of the involved spinal motor neurons. The DCTR sequence is able to recruit cMAP in a similar manner to recruiting of neuromuscular junctions through a series of incremental peaks. The obtained cMAP is smaller than the cMAP that is obtainable by means of the traditional neurophysiological modes with a pulse width of >100 ms. With regard to the aforesaid recruitment of the cMAPs through FREMS stimulation, the presence of a linear trend in the increase of the cMAP must also be emphasised that is coherent with the incremental trend of width and frequency of the DCTR sequence. Actually, more than the single variations of f and w it is the w/f ratio that better discloses the contribution of both variables to the intensity of the stimulus. Further, it can be found that the correlation between the w/f ratio and the amplitude of the H reflex is not of linear type. It can thus be stated that the amplitude of the cMAP is determined not only by the intensity of the stimulus but that also the temporal stimulation sequence has great relevance. By applying the transcutaneous electrodes of the apparatus Lorenz™ directly on the adductor hallucis muscle, the stimulation near the muscle certainly not being identical to the stimulation of a motor nerve, it has been demonstrated that this mode of administration below the motor threshold, but sequentially ordered, is able to influence the excitability of the spinal motor neurons.

With reference to FIGS. 8 to 11, during the subphase C of all the sampled FREMS stimulation cycles, a strong linear correlation can be found between the amplitude of the H reflex and the w/f ratio, (r²=0.43; P<0.001). As previously mentioned, one of the most important systems for regulating the spinal excitability is the reflex path that originates from the muscle spindles and influences the excitability of the pool of the alpha motor neurons by means of the inhibitory interneurons. It is supposed that an electric recruitment of muscular activity may, at a low stimulation intensity, be more effective in activating muscle spindles rather than the entire striated muscle following the low activation threshold of the muscle spindles. In the absence of FREMS stimulation, the amplitude of the H reflex shows a spontaneous and progressive attenuation due to a traditional accommodation mechanism. On the other hand, during FREMS stimulation, the trend of the amplitude of the H reflex is greatly attenuated and in a constant manner. The phase B of the DCTR sequence is in fact distinguished by the increase in the width of the constant frequency pulses; this is a “tonic” and proportional activation mode to which the nuclear bag muscle spindles are more sensitive. It can be supposed that the trend of the H reflex during the subphase B of the DCTR sequence is an expression of a prevalent involvement of nuclear-bag spindles. During the subphase C, on the other hand, rapid and reproducible oscillations of the amplitude of the H reflex occur, in linear correlation with the rapid frequency increase of the pulses of the DCTR sequence. The nuclear chain muscle spindles are preferentially activated by high-frequency and high-variability stimuli. On the basis of the foregoing remarks, it can be supposed that the phase C of the DCTR sequence is preferably active on the contingent of the nuclear chain muscle spindles. In the terminal phase of the phase C the amplitude of the H reflex again shows an increase although the stimulation frequency reaches the maximum value. This is the effect of the stimulation of the Ib receptors due to the tendon stretching during the contraction of the muscle. Another fundamental physiological implication of this analysis is that the effect induces a significant persistence of the attenuation of the average of the H reflex even after the end of the DCTR stimulation. This persistency in suppressing the amplitude of the reflex reflects an adaptational increase of the spinal inhibitory activity that has never been shown in the literature.

Since this has highlighted the possibility of devising new therapies for certain motion disorders that are distinguished by an abnormal motor neuron excitability, the aforesaid hypotheses were subjected to clinical experimentation. The latter was conducted on hospitalized patients suffering from pathologies of the upper motor neuron, such as haemiplegia, paraplegia, quadriplegia or spastic tetraparesis. These pathologies were a consequence of the ischemic phenomena, central haemorrhagic (brain stroke or head injury) phenomena or spinal cord lesions.

The therapeutic protocol consisted of simultaneously stimulating the hypertonic muscle with DCTR sequences and the antagonist muscle with ATMC sequences. Reasonably alert patients having a reasonable sense of space and time and a decent or high degree of cooperation, not suffering from fixed contractions of the joints and from grade 2-4 muscle-tendon retractions on the modified Rankin Scale (mRS), were accepted for treatment. On the other hand, patients having an altered state of consciousness, patients who were not very or not at all cooperative, wearers of pacemakers or implantable defibrillators, and patients affected by pathologies that were such as not to allow the use of electrotherapies, were excluded. The patients were assessed clinically at the moment of recruitment, at the end of the treatment and at 15, 30 and 45 days from the end of the therapy. For the functional assessments specific clinical scales were used: Ashworth Scale, A.D.L. Index (Activities of Daily Living according to Barthel), Rankin Scale, Spasm Frequency Scale, Motricity Index, FIM (Functional Independence Measure). These clinical scales enable the degree of tone and spasticity of a patient to be assessed and the possibility of the latter to perform motor functions with the limbs, to walk independently and to be independent in activities of daily living (ADL). For the pain assessment, the VAS 0-100 scale was used. The patients were subjected to a daily treatment session for 15 consecutive sessions. At the initial assessment all the patients had a grade 2 Ashworth spastic hypertonia of the lower limbs. At the end of the first cycle of therapy a reduction of hypertonia was found, with a grade 1 Ashworth average assessment. These evidences show the clinical efficacy of the method and of the electrostimulating apparatus that have been previously disclosed.

1-83. (canceled)

84. Electrostimulating apparatus, comprising a generating arrangement for generating electric pulses organised in sequences having preset values of typical parameters, said typical parameters comprising amplitude, width and frequency of said pulses, a plurality of stimulation channels such as to dispense said sequences to body zones of an organism in an independent manner, a varying arrangement suitable for varying at least one of said typical parameters so as to substantially prevent said organism from habituating to said electric pulses.

85. Apparatus according to claim 84, wherein said sequences comprise relaxing and/or vasoactive sequences.

86. Apparatus according to claim 84, wherein said varying arrangement comprises a timing device arranged for varying said width of said pulses.

87. Apparatus according to claim 86, wherein said timing device is further arranged for varying said frequency of said pulses.

88. Apparatus according to claim 86, wherein said timing device comprises an integrated timing unit.

89. Apparatus according to claim 84, wherein said varying arrangement comprises a digital-analogue converter arranged for varying said amplitude of said pulses.

90. Apparatus according to claim 84, comprising a remote-control device arranged for varying said amplitude of said pulses.

91. Apparatus according to claim 90, wherein said varying arrangement comprises a digital-analogue converter arranged for varying said amplitude of said pulses and said remote-control device acts, in use, on said digital-analogue converter.

92. Apparatus according to claim 88, wherein said integrated timing unit is able to produce a percentage increase of said width of said pulses, said pulses being performed in a variable number of phases.

93. Apparatus according to claim 92, wherein said percentage increase is correlated to a number of phase, to an initial stimulation pulse width and to a stimulation pulse width expressed in function of said number of phase, according to the following formula:
Tᵢ(Nf)=T₀×(1+I%)^{Nf}

94. Apparatus according to claim 92, wherein said percentage increase has a value that is selected from a group of values comprising: 20%, 25%, 33%, 50%.

95. Apparatus according to claim 93, wherein said number of phase is selected from a group of values comprising: 0, 1, 2, 3, 4, 5, 6, 7, 8, 9.

96. Apparatus according to claim 93, wherein said integrated timing unit is able to vary in a pseudorandom manner an interval of time comprised between two consecutive phases of said phases, this enabling to vary said width of said pulses in a random manner and to substantially prevent said organism from habituating to said pulses.

97. Apparatus according to claim 84, wherein said plurality of stimulation channels comprises four stimulation channels.

98. Apparatus according to claim 84, wherein said plurality of stimulation channels comprises a transcutaneous electrode arrangement.

99. Apparatus according to claim 98, wherein said transcutaneous electrode arrangement comprises a plurality of pairs of transcutaneous electrodes.

100. Apparatus according to claim 16, wherein said plurality of pairs of transcutaneous electrodes comprises four pairs of transcutaneous electrodes.

101. Apparatus according to claim 98, wherein said plurality of stimulation channels comprises a plurality of outlet selecting devices to which said transcutaneous electrode arrangement is connected electrically.

102. Apparatus according to claim 101, wherein said plurality of outlet selecting devices comprises a plurality of pairs of outlet selectors.

103. Apparatus according to claim 102, wherein said plurality of pairs of outlet selectors is provided with a plurality of switches arranged for alternatively activating and/or deactivating said transcutaneous electrode arrangement.

104. Apparatus according to claim 101, wherein said plurality of outlet selecting devices is connected electrically to said pulse generating arrangement.

105. Apparatus according to claim 84 wherein, in use, said pulse generating arrangement receives a voltage comprised between 0 and 300 Volt, said voltage being produced by a voltage adjusting device.

106. Apparatus according to claim 88, wherein said integrated timing unit pilots said pulse generating arrangement.

107. Apparatus according to claim 84, further comprising a control unit arranged for piloting, in use, said plurality of stimulation channels and such as to be able to alternatively activate and/or deactivate in an independent manner each channel of said plurality, so as to enable a desired number of said body zones to which to dispense said pulses to be selected.

108. Apparatus according to claim 107, wherein said timing device comprises an integrated timing unit and said integrated timing unit is comprised in said control unit.

109. Apparatus according to claim 107, wherein said control unit comprises an analogue-digital converter, arranged for receiving a plurality of feed-back signals relating to said amplitude of said pulses.

110. Apparatus according to claim 107, wherein said varying arrangement comprises a digital-analogue converter arranged for varying said amplitude of said pulses and, in use, said pulse generating arrangement receives a voltage comprised between 0 and 300 Volt, said voltage being produced by a voltage adjusting device and said control unit pilots said voltage adjusting device through said digital-analogue converter.

111. Apparatus according to claim 107, further comprising a timing control device arranged for controlling said width and said frequency of said pulses and able to produce a width error signal and/or a frequency error signal such as to stop said control unit.

112. Apparatus according to claim 107, wherein, in said control unit there can be housed a support readable through a data processing device, said support containing a plurality of data defining said typical parameters of said sequences and being arranged for piloting said apparatus.

113. Apparatus according to claim 84, further comprising a further control unit functionally associated with an alphanumeric keyboard and with a display, said alphanumeric keyboard and said display being arranged for controlling, in use, the operation of said apparatus (1).

114. Apparatus according to claim 113, further comprising a control unit arranged for piloting, in use, said plurality of stimulation channels and such as to be able to alternatively activate and/or deactivate in an independent manner each channel of said plurality, so as to enable a desired number of said body zones to which to dispense said pulses to be selected, wherein said control unit and said further control unit mutually interact through a serial communication interface.

115. Apparatus according to claim 113, wherein, in said control unit there can be housed a support readable through a data processing device, said support containing a plurality of data defining said typical parameters of said sequences and being arranged for piloting said apparatus and said support can be housed in said further control unit.

116. Apparatus according to claim 84, wherein, in use, said pulses are produced in said plurality of stimulation channels in such a way that said frequency is the same in said pulses.

117. Apparatus according to claim 84, wherein, in use, said pulses are produced in said plurality of stimulation channels in such a way that said frequency is made different in said pulses.

118. Apparatus according to claim 84, wherein, in use, said pulses are dispensed in a simultaneous manner through said plurality of stimulation channels.

119. Apparatus according to claim 84, wherein, in use, said pulses are dispensed in a sequential manner through said plurality of stimulation channels.

120. Apparatus according to claim 84, wherein, in use, said pulses are dispensed in a simultaneous manner to a variable number of said body zones, said number being selected from a group comprising: 2, 4, 8, 16.

121. Apparatus according to claim 84, wherein, in use, said pulses are dispensed in a sequential manner to a variable number of said body zones, said number being selected from a group comprising: 2, 4, 8, 16.

122. Apparatus according to claim 85, wherein said relaxing and/or vasoactive sequences are based on said width, said frequency and on intervals of time during which a plurality of combinations between said width and said frequency is produced.

123. Apparatus according to claim 122, wherein said relaxing sequences comprise a muscle fibre relaxing sequence, comprising a subphase, wherein said frequency and said width are constant, a further subphase, wherein said frequency is constant and said width is variable, a still further subphase, wherein said frequency is variable and said width is constant.

124. Apparatus according to claim 122, wherein said vasoactive sequences comprise a microcircle activating sequence, comprising a subsequence, a further subsequence and a still further subsequence, in said subsequence and in said still further subsequence there being produced an increase of said frequency, in said further subsequence said width being prevalently varied.

125. Method for electrostimulating an organism, comprising:
producing a sequence of electric pulses having a relaxing effect and further producing a further sequence of electric pulses having a vasoactive effect;
dispensing said sequence to body zones of said organism, and further dispensing said further sequence to further body zones of said organism, said body zones and said further body zones comprising an agonist muscle and an antagonist muscle of a neuromuscular compartment comprised in said organism.

126. Method according to claim 125, wherein said agonist muscle and said antagonist muscle are comprised in distinct neuromuscular districts of a motor limb of said organism.

127. Method according to claim 125, wherein said body zones and said further body zones are mutually connected through at least one nervous circuit comprising an afferent neuron, an interneuron and an alpha-type motor neuron.

128. Method according to claim 125, wherein said producing and said further producing comprise using an electrostimulating apparatus.

129. Method according to claim 125, comprising varying at least one of typical parameters of said pulses, said typical parameters comprising amplitude, width and frequency.

130. Method according to claim 129, wherein said producing and said further producing comprise using an electrostimulating apparatus and said varying comprises using varying arrangement comprised in said electrostimulating apparatus.

131. Method according to claim 130, wherein said varying comprises varying said width of said pulses through a timing device comprised in said varying devices.

132. Method according to claim 131, wherein said varying comprises varying said frequency of said pulses through said timing device.

133. Method according to claim 131, wherein said varying comprises using an integrated timing unit comprised in said timing device.

134. Method according to claim 130, wherein said varying comprises varying said amplitude of said pulses through a digital-analogue converter comprised in said varying arrangement.

135. Method according to claim 134, wherein said varying said amplitude comprises using a remote-control device that is able to act on said digital-analogue converter.

136. Method according to claim 129, comprising producing a percentage increase in said width of said pulses, said pulses being performed in a variable number of phases.

137. Method according to claim 136, wherein said percentage increase is correlated to a number of phase, to an initial stimulation pulse width and to a stimulation pulse width expressed in function of said number of phase, according to the following formula:
Tᵢ(Nf)=T₀×(1+I%)^{Nf}

138. Method according to claim 136, wherein a value of said percentage increase is selected from a group of values comprising: 20%, 25%, 33%, 50%.

139. Method according to claim 137, wherein said number of phase is selected from a group of values comprising: 0, 1, 2, 3, 4, 5, 6, 7, 8, 9.

140. Method according to claim 136, wherein said varying comprises using an integrated timing unit comprised in said timing device and said producing said percentage increase is obtained through said integrated timing unit.

141. Method according to claim 136, comprising varying in a pseudorandom manner an interval of time comprised between two consecutive phases of said phases, this enabling to vary said width of said pulses in a random manner and to substantially prevent said organism from habituating to said pulses.

142. Method according to claim 141, wherein said varying comprises using an integrated timing unit comprised in said timing device and said varying in a pseudorandom manner is obtained through said integrated timing unit.

143. Method according to claim 128, wherein said dispensing and said further dispensing are obtained through a plurality of stimulation channels comprised in said electrostimulating apparatus.

144. Method according to claim 143, wherein said dispensing and said further dispensing comprises using a transcutaneous electrode arrangement comprised in said plurality of stimulation channels.

145. Method according to claim 144, wherein said dispensing and said further dispensing comprises using a plurality of pairs of transcutaneous electrodes comprised in said transcutaneous electrode arrangement.

146. Method according to claim 144, comprising alternatively activating and/or deactivating said transcutaneous electrode arrangement through a plurality of outlet selecting device comprised in said electrostimulating apparatus and electrically connected to said transcutaneous electrode arrangement.

147. Method according to claim 146, wherein said alternatively activating and/or deactivating said transcutaneous electrode arrangement comprises acting on a plurality of switches comprised in said outlet selecting device.

148. Method according to claim 146, wherein said producing and said further producing comprise using pulse generating arrangement, comprised in said electrostimulating apparatus and connected electrically to said outlet selecting device.

149. Method according to claim 148, comprising producing a voltage comprised between 0 and 300 Volt through a voltage adjusting device and sending said voltage to said pulse generating arrangement.

150. Method according to claim 148, comprising varying at least one of typical parameters of said pulses, said typical parameters comprising amplitude, width and frequency, wherein said varying comprises using an integrated timing unit comprised in said timing device and the method comprising piloting said pulse generating arrangement through said integrated timing unit.

151. Method according to claim 143, comprising piloting said plurality of stimulation channels in such a way as to be able to alternatively activate and/or deactivate in an independent manner each channel of said plurality, so as to enable a desired number of said body zones and/or of said further body zones to which to dispense and/or further dispense said pulses to be selected.

152. Method according to claim 151, wherein said piloting said plurality of stimulation channels is obtained through a control unit comprised in said electrostimulating apparatus.

153. Method according to claim 128, comprising piloting said electrostimulating apparatus through a support readable through a data processing device, said support containing a plurality of data defining typical parameters of said sequences and/or said further sequences.

154. Method according to claim 153, wherein said piloting said electrostimulating apparatus comprises using an alphanumeric keyboard and a display comprised in said electrostimulating apparatus.

155. Method according to claim 143, comprising varying at least one of typical parameters of said pulses, said typical parameters comprising amplitude, width and frequency, wherein said producing and/or said further producing said pulses in said plurality of stimulation channels occurs in such a way that said frequency is the same in said pulses.

156. Method according to claim 143, comprising varying at least one of typical parameters of said pulses, said typical parameters comprising amplitude, width and frequency, wherein said producing and/or said further producing said pulses in said plurality of stimulation channels occurs in such a way that said frequency is different in said pulses.

157. Method according to claim 143, wherein said dispensing and/or said further dispensing said pulses occurs in a simultaneous manner through said plurality of stimulation channels.

158. Method according to claim 143, wherein said dispensing and/or said further dispensing said pulses occurs in a sequential manner through said plurality of stimulation channels.

159. Method according to claim 125, wherein said dispensing and/or further dispensing said pulses occurs in a simultaneous manner at a variable number of said body zones and/or of said further body zones, said number being selected from a group comprising: 2, 4, 8, 16.

160. Method according to claim 125, wherein said dispensing and/or further dispensing said pulses occurs in a sequential manner at a variable number of said body zones and/or of said further body zones, said number being selected from a group comprising: 2, 4, 8, 16.

161. Method according to claim 129, wherein said sequence having a relaxing effect and/or said further sequence having a vasoactive effect are based on said width, said frequency and on intervals of time during which there is produced a plurality of combinations between said width and said frequency.

162. Method according to claim 161, wherein said sequence having a relaxing effect comprises a subphase, wherein said frequency and said width are constant, a further subphase, wherein said frequency is constant and said width is variable, a still further subphase, wherein said frequency is variable and said width is constant.

163. Method according to claim 161, wherein said sequence having a vasoactive effect is a microcircle activating sequence, comprising a subsequence, a further subsequence and a still further subsequence, in said subsequence and in said still further subsequence there being produced an increase of said frequency, in said further subsequence said width being prevalently varied.

164. Method according to claim 125, wherein said dispensing and said further dispensing enable a hypertonic contraction of said agonist muscle to be substantially inhibited.

165. Method according to claim 164, wherein said inhibiting said hypertonic contraction is obtained by producing a phase depression of the H reflex.

166. Support readable through a data processing device, carrying a plurality of data defining typical parameters of sequences of electric pulses, usable in combination with an electrostimulating apparatus according to claim 84.
